(19) United States
(12) Patent Application Publication
(10) Pub. No.: US 2022/0061796 A1
(43) Pub. Date: Mar. 3, 2022

(54) DISPOSABLE STETHOSCOPE

(71) Applicant: Wichita State University, Wichita, KS (US)

(72) Inventors: Kenneth Gardner, Wichita, KS (US); Kevin Gaines, Wichita, KS (US); William Smith, Wichita, KS (US); Joel White, Wichita, KS (US)

(21) Appl. No.: 17/446,682

(22) Filed: Sep. 1, 2021

Related U.S. Application Data
(60) Provisional application No. 63/073,071, filed on Sep. 1, 2020.

(51) Int. Cl. A61B 7/02; A61B 5/00

(52) U.S. Cl. CPC A61B 7/02; A61B 5/6814; A61B 5/6823

(57) ABSTRACT

A disposable stethoscope comprising a headset and a chestpiece assembly configured to be acoustically connected to the headset for conveying sound to the headset. The chestpiece assembly comprises a diaphragm, a chestpiece body, and a retention ring. The chestpiece body comprises a drum including a peripheral annular rim and a central hole. The diaphragm comprises a peripheral edge margin configured to be positioned on the drum such that the peripheral edge margin of the diaphragm is supported on the rim. The retention ring comprises a ring portion extending circumferentially about a ring axis and at least one latch arm extending along the ring axis. The retention ring is configured to be positioned on the chestpiece body to sandwich the peripheral edge margin of the diaphragm between the rim and the ring portion and the arms latch with the chestpiece body to attach the retention ring to the chestpiece body.

### CROSS-REFERENCE TO RELATED APPLICATION

The present application claims priority to the U.S. Provisional Patent Application No. 63/073,071 filed on Sep. 1, 2020, which is hereby incorporated by reference.

### FIELD

The present disclosure generally relates to acoustic stethoscopes.

### BACKGROUND

Acoustic stethoscopes are used for monitoring a patient's physiological processes. This act of listening to the sounds a patient's heart, lungs, or other organs such as those located in the abdomen is commonly called auscultation. Generally, acoustic stethoscopes transmit sound into a chestpiece, through hollow tubes, to a binaural headpiece worn by a nurse, doctor or other practitioner. The chestpiece is placed on (e.g., in contact with) the patient's body. Conventionally, the chestpiece includes a diaphragm that vibrates in response to the sounds of the patient's body. Doctors and nurses commonly use reusable stethoscopes on multiple patients in the course of a workday. And in many cases, stethoscopes are not sterilized between patients. Occasionally, the same stethoscope may even be exchanged among different practitioners without being sterilized first. This, of course, poses a potential health risk to both the patient and the practitioner, providing a vector for surface-borne viruses to reach new, sometimes acutely vulnerable, hosts. Disposable stethoscopes are a known solution to this problem, but existing options offer sub-par acoustic performance, and as a result, have not been widely adopted.

### SUMMARY

In one aspect, a disposable stethoscope is disclosed. The stethoscope comprises a headset and a chestpiece assembly configured to be acoustically connected to the headset for conveying sound to the headset. The chestpiece assembly comprises a diaphragm, a chestpiece body, and a retention ring. The chestpiece body comprising a drum having a front side and a rear side spaced apart along a drum axis. The front side of the drum includes a peripheral annular rim. The drum comprises a central hole opening through the front side of the drum. The diaphragm comprises a peripheral edge margin, the diaphragm being configured to be positioned on the front side of the drum such that the peripheral edge margin of the diaphragm is supported on the rim. The retention ring comprises a ring portion extending circumferentially about a ring axis and at least one latch arm extending along the ring axis to a respective free end portion. The retention ring is configured to be positioned on the chestpiece body such that the retention ring sandwiches the peripheral edge margin of the diaphragm between the rim and the ring portion. At least one arm latches with the chestpiece body to attach the retention ring to the chestpiece body and thereby retain the diaphragm on the drum.

In another aspect, a disposable stethoscope is disclosed comprising a chestpiece, a first and second eartips, and a headset configured to acoustically connect the chestpiece to the first and second eartips for transmitting sound from the chestpiece to the first and second eartips. The headset comprises a generally wye-shaped tubing including a stem tube, a first binaural tube, and a second binaural tube. The stem tube has a lower end portion and an upper end portion. The first binaural tube extends upward from the upper end portion of the stem tube in a first direction to a first binaural end portion. The second binaural tube extends upward from the upper end portion of the stem tube in a second direction to a second binaural end portion. Each of the first and second binaural end portions defining an eartip mount configured to mount a respective one of the first and second eartips on the headset. The headset further comprises a brace extending laterally from a first brace end portion to a second end brace portion such that the brace is configured to support the first and second binaural tubes in laterally spaced apart relation. The first end brace portion is connected to the first binaural tube at a location spaced apart between the upper end portion of the stem tube and the first binaural end portion. The second brace end portion is connected to the second binaural tube at a location spaced apart between the upper end portion of the stem tube and the second binaural end portion. The headset is formed from a single piece of monolithic material.

In another aspect, a disposable stethoscope is disclosed comprising a chestpiece, a first and second eartips, and a headset configured to acoustically connect the chestpiece to the first and second eartips for transmitting sound from the chestpiece to the first and second eartips. The headset comprising generally wye-shaped tubing including a stem tube, a first binaural tube, and a second binaural tube. The stem further includes a lower end portion and an upper end portion. The first binaural tube extends upward from the upper end portion of the stem tube in a first direction to a first binaural end portion. The second binaural tube extends upward from the upper end of the stem tube in a second direction to a second binaural end portion. Each of the first and second binaural end portions defines an eartip mount configured to mount a respective one of the first and second ear tips on the headset. The headset defines passaging providing communication from the lower end portion of the stem to each of the first and second binaural end portions. The passaging includes a stem lumen, a first binaural lumen, and a second binaural lumen. The stem lumen extends along the stem from a lower end portion opening through the lower end portion of the stem to an upper end portion opening through the upper end portion of the stem. The first binaural lumen extends along the first binaural tube from a lower end portion connected to the upper end portion of the stem lumen to an upper end portion opening through the eartip mount of the first binaural tube. The second binaural lumen extends along the second binaural tube from a lower end portion connected to the upper end portion of the stem lumen to an upper end portion opening through the eartip mount of the second binaural tube. Each of the first and second binaural lumens has a cross-sectional dimension that tapers as the respective binaural lumen extends upward from adjacent the respective lower end portion toward the respective upper end portion.

Other objects and features of the present disclosure will be in part apparent and in part pointed out herein.

### BRIEF DESCRIPTION OF THE DRAWINGS

FIG. 1 is a front view of a stethoscope;

FIG. 2 is a front view of a headpiece of a stethoscope;

FIG. 2A is a cross section of the headpiece showing internal passaging thereof;

FIG. 3 is a perspective of the headpiece;

FIG. 4 is a printed perspective of the stethoscope;

FIG. 5 is a side view of the headpiece;

FIG. 6 is a bottom view of the headpiece;

FIG. 7 is a perspective of a chestpiece body of the stethoscope;

FIG. 8 is a perspective of a retention ring of the stethoscope;

FIG. 9 is a front view of a chestpiece including the chestpiece body and retention ring.

FIG. 10 is a rear view of the chestpiece;

FIG. 11 is a perspective of another embodiment of the stethoscope;

FIG. 12 is a perspective of still another embodiment of a headpiece of a stethoscope; and

FIG. 13 is an exploded perspective of another embodiment of the chestpiece.

Corresponding reference characters indicate corresponding parts throughout the drawings.

### DETAILED DESCRIPTION

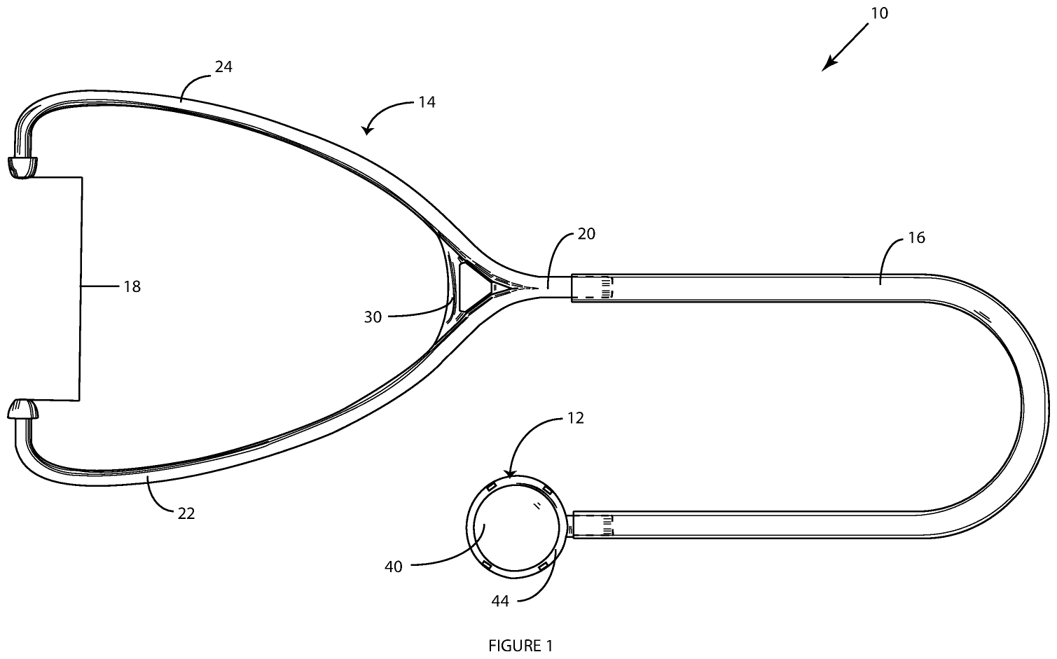

Referring to FIG. 1, a stethoscope is disclosed, generally indicated at reference number 10. The stethoscope 10 generally comprises a chestpiece 12, a headset 14, a flexible extension tube 16 which connects the chestpiece to the headset, and a pair of eartips 18 configured to operably connect the binaural ends of the headset in the ears of a wearer for auscultation. As will be explained in further detail below, the illustrated stethoscope 10 can be constructed relatively inexpensively such that it is economical to use the stethoscope as a one-time-use or disposable product. However, in comparison with existing disposable stethoscope solutions available on the market, it is believed that stethoscope 10 may have improved acoustic performance.

In general, the headset 14 is configured to acoustically connect the chestpiece 12 to the first and second eartips 18 for transmitting sound from the chestpiece to the eartips. The illustrated headset 14 comprises generally wye-shaped tubing including a stem tube 20 having a lower end portion and an upper end portion, a first binaural tube 22 extending upward from the upper end portion of the stem tube in a first direction to a first binaural end portion, and a second binaural tube 24 extending upward from the upper end portion of the stem tube in a second direction to a second binaural end portion. The lower end portion of the stem 20 is tapered or beveled to facilitate insertion of the stem into the lower end portion of the extension tube 16 to attach a chestpiece body 42 of the chestpiece 12 to the extension tube by friction fit.

Throughout this disclosure terms describing relative vertical positions (e.g., upper, lower, top, bottom) are understood to refer to the orientation of the stethoscope 10 when suspended on the ears or neck of a wearer in an upright position. In other words, the terms of relative vertical position are used as if the stethoscope 10 is supported so that the chestpiece 12 generally defines the bottom end portion of the stethoscope and the eartips 18 generally define the top end portion of the stethoscope. It will be understood, however, that the orientation of the stethoscope may vary during use. For example, the stethoscope may become inverted at times.

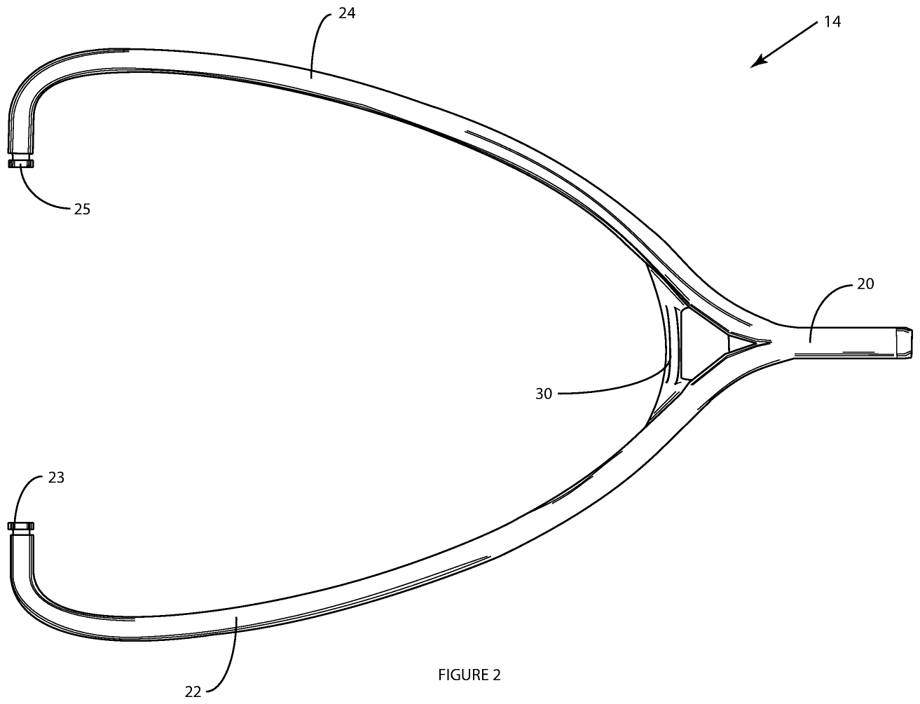

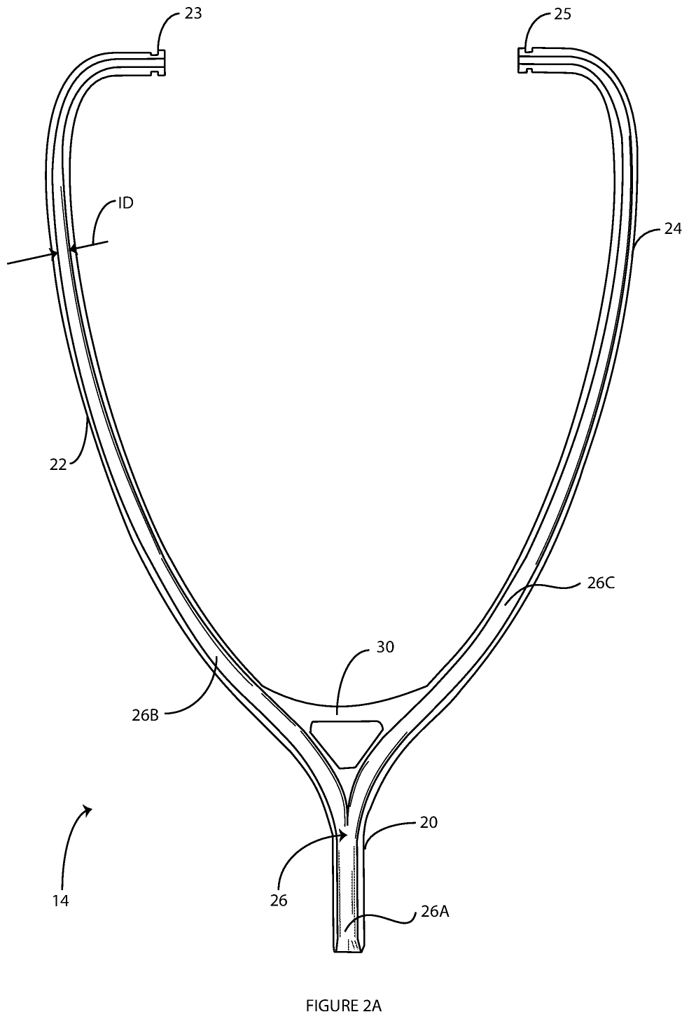

Referring to FIG. 2, each of the first and second binaural end portions defines a respective first and second eartip mount 23, 25 configured to mount a respective one of the first and second eartips on the headset 14. In the illustrated embodiment, each eartip mount 23, 25 comprises an annular channel or groove, and each eartip 18 comprises a mating tongue or lip (not shown) configured to snap into the groove to retain the eartip on the respective binaural tube 22, 24. Other ways of mounting eartips on the binaural headset may also be used without departing from the scope of the disclosure. In general, each eartip 18 may comprise a hollow piece of silicone shaped and arranged for being seated in the ear of the medical practitioner in a known manner.

Referring to FIG. 2A, the headset defines passaging 26 providing fluid communication from the lower end portion of the stem 20 through each of the first and second binaural tube end portions. The passaging 26 further includes a stem lumen 26a, a first binaural lumen 26b, and a second binaural lumen 26c. The stem lumen 26a extends along the stem 20 from the lower end portion of the stem to the upper end portion of the stem. The lower end portion of the stem lumen 26a opens through the lower end portion of the stem 20, and the upper end portion of the stem lumen opens through the upper end portion of the stem. The first binaural lumen 26b extends along the first binaural tube 22 from a lower end portion of the first binaural tube to the upper end portion of the first binaural tube. A lower end portion of the first binaural lumen 26b connects to the upper end portion of the stem lumen 26a, while the upper end portion of the first binaural lumen opens through the first eartip mount 23 at the upper end portion of the first binaural tube 22. The second binaural lumen 26c extends along the second binaural tube 24 from a lower end portion of the second binaural tube to and upper end portion of the second binaural tube. A lower end portion of the second binaural lumen opens to the upper end portion of the stem lumen 26a, while an upper end portion of the second binaural lumen opens through the second eartip mount 25 at the upper end portion of the first binaural tube 24.

Each of the binaural lumens 26b, 26c comprises a cross-sectional dimension ID (e.g., the inner diameter of the respective binaural tube 22, 24). In an exemplary embodiment, the binaural tubes 22, 24 are configured so that the cross-sectional dimension ID tapers or gradually decreases as the binaural lumen 26b, 26c extends upward from a location adjacent the upper end portion of the stem lumen 26a toward the upper end portion of the respective binaural lumen. Thus, the cross-sectional area in planes perpendicular to the center axis of each binaural lumen 26b, 26c may gradually decrease in the upward direction along the length of the respective binaural lumen (e.g., the cross-sectional dimension and/or cross-sectional area may gradually decrease along a portion or all of the length of the respective binaural lumen).

The headset 14 further comprises a brace 30 extending laterally from a first brace end portion connected to the first binaural tube 22 to a second brace end portion connected to the second binaural tube 24. The first brace end portion is located between the upper end portion of the stem 20 and the upper end portion of the first binaural tube 22, while the second brace end portion is located between the upper end portion of the stem and the upper end portion of the second binaural tube 24. For example, in the illustrated embodiment, the brace 30 is located along the lengths of the respective binaural tubes 22, 24 at a location closer to the stem 20 than to the upper end portions of the binaural tubes. Suitably, the brace 30 may be a solid body devoid of internal lumens or openings as shown in FIG. 2A. The illustrated brace is configured to support the first and second binaural tubes 22, 24 in laterally spaced apart relation. More particularly, the brace 30 is configured to hold the binaural tubes 22, 24 so that they remain laterally spaced apart but do not spread apart by an excessive amount. Thus, it can be seen that, in the illustrated embodiment, the brace 30 replaces the binaural spring of a conventional acoustic stethoscope. Eliminating the binaural spring is thought to reduce the cost of production and simplify manufacture of the stethoscope 10 by minimizing the required number of parts.

In one embodiment, the headset 14 is formed from a single piece of monolithic material. In other words, the stem 20, the binaural tubes 22, 24, and the brace 30 are all formed from one monolithic and unitary piece of material. Suitable materials for the headset 14 will be workable in manufacturing processes that are capable of forming the one-piece headset, for example, materials capable of being formed in the required shape in an injection molding process or an additive manufacturing process. The material chosen also preferably has acoustic properties that enable conveyance of sound waves through the passaging 26 from the chestpiece 12 and extension tube 16 to the eartips 18. In one embodiment, the headset 14 is formed from one of a PLA, an ABS, a nylon, or a PVA.

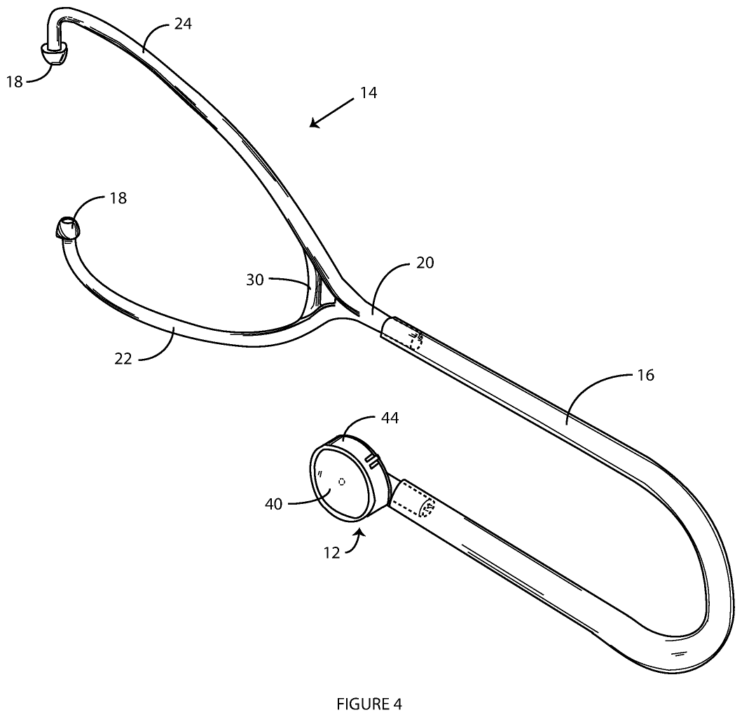

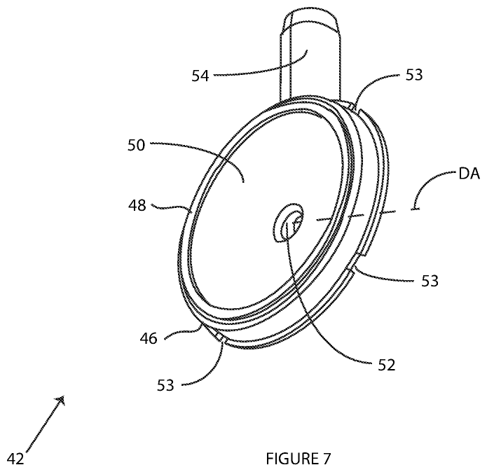

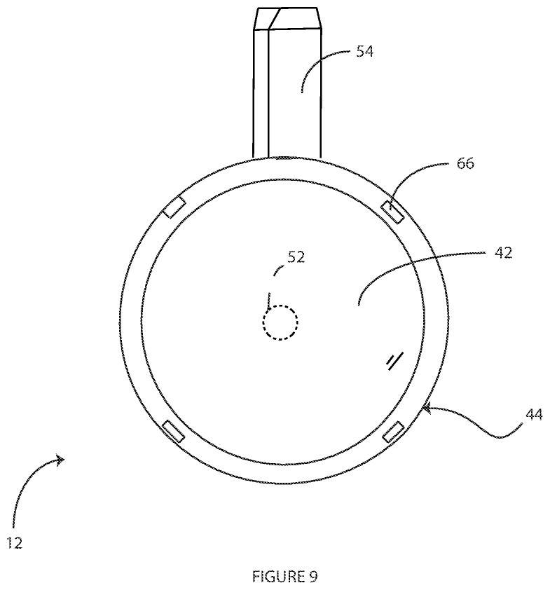

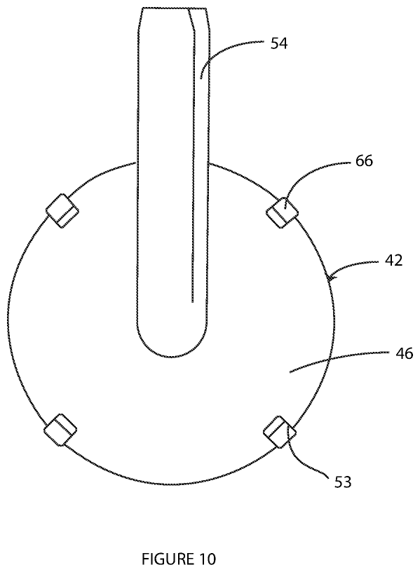

Referring to FIGS. 1, 4, and 7-10, the chestpiece 12 comprises a multipart chestpiece assembly comprising a diaphragm 40, the chestpiece body 42, and a retention ring 44. The chestpiece body comprises a drum 46 having a front side and a rear side spaced apart along a drum axis DA (FIG. 7). The front side of the drum includes a peripheral annular rim 48 and a bell-shaped or conical central portion 50 radially inboard of the rim 48 with respect to the drum axis DA. The drum 46 comprises a central hole 52 opening through the front side of the drum at about the center or most recessed area of the conical portion 50. The illustrated central hole 52 extends rearward along the axis DA, along a portion of the thickness of the drum 46 but does not penetrate the rear side of the chestpiece body 42. Thus, in the illustrated embodiment, the chestpiece 12 is a single-sided chestpiece. However, it will be understood that double-sided or turnable chestpieces can also be used without departing from the scope of the disclosure.

In the illustrated embodiment, the rear side of the drum 46 comprises a perimeter edge margin that defines a plurality of notches 53 at angularly spaced apart locations about the drum axis DA. As will be explained in further detail below, the notches 53 are configured to help secure the retention ring 44 to the chestpiece body 42, in particular, to limit rotation of the retention ring 44 relative to the chestpiece body 42 about the drum axis DA.

The illustrated chestpiece body 42 further comprises a stem 54 extending radially outward (e.g., upward) from the drum 46 with respect to drum axis DA. A radial lumen (not shown) extends from an inner end portion connected to the rear end portion of the hole 52 to an upper end portion that opens through an outer or upper end portion of the stem 54. Suitably, the upper end portion of the stem 54 is tapered or beveled to facilitate insertion of the stem into the lower end portion of the extension tube 16 to attach the chestpiece body 42 to the extension tube by friction fit.

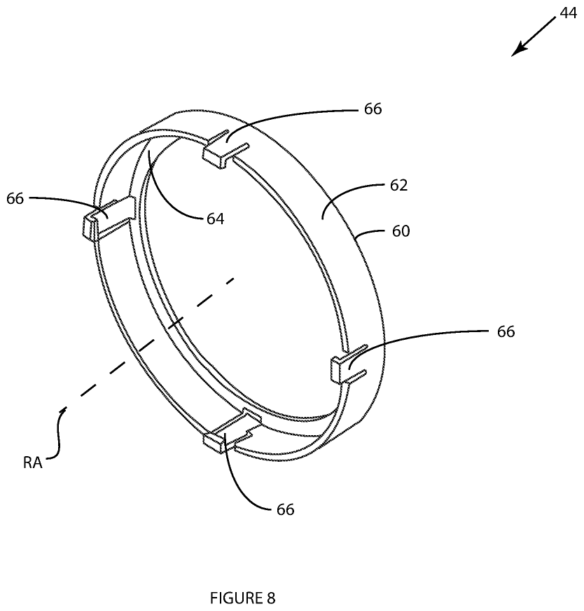

Referring to FIG. 8, the retention ring 44 comprises a ring portion 60 extending circumferentially about a ring axis RA. In the illustrated embodiment the ring portion 60 includes a collar section 62 that has a generally cylindrical shape extending lengthwise along the ring axis RA and a lip section 64 that extends radially inward with respect to the ring axis from a front edge of the collar section 62. The illustrated collar section 62 includes a plurality of latch arms 66 at circumferentially spaced apart locations about the ring axis RA. Each latch arm 66 extends along the ring axis RA from an attached end portion adjacent the lip section 64 to a free end portion that defines a radially inwardly protruding latch hook. In the illustrated embodiment, each latch arm 66 is located within a respective cutout portion of the collar section 62. In one or more embodiments, each latch arm 66 is resiliently bendable radially outward with respect to a remainder of the collar section 62.

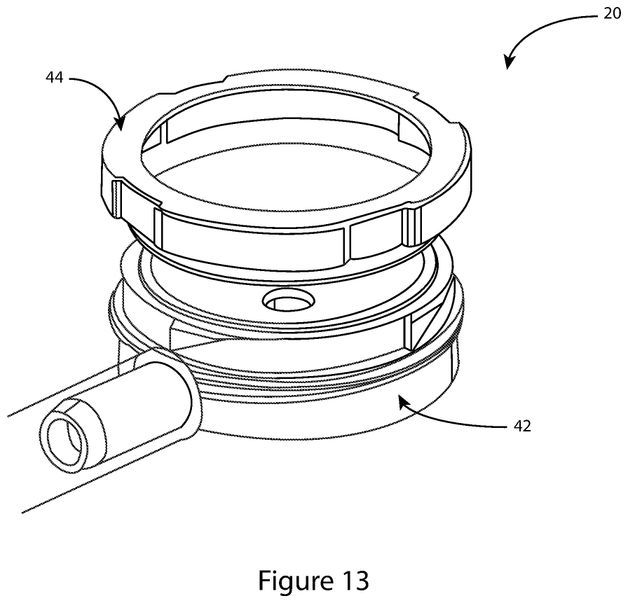

The retention ring 44 is configured to be mounted on the front side of the drum 46 such that the lip section 64 is located in front of the rim portion 48 of the drum and overlaps the rim portion. In addition, the collar section 62 extends circumferentially around the side wall of the drum 46. Each latch arm 66 is configured to be received in a respective notch 53 such that the inwardly protruding latch hooks protrude inward along the rear side of the drum 46 and thereby latch onto the drum. Thus, it can be seen that, to (releasably) attach the retention ring 44 to the chestpiece body 42, the assembler positions the retention ring 44 in front of the front side of the drum 46 with the latch arms 66 pointing rearward in rotational registration with the notches 53. Then the assembler pushes the retention ring 44 rearward and deflects the latch arms 66 radially outward until the inwardly protruding latch hooks clear the rear side of the drum 46. At this point, the latch arms 66 resiliently rebound into the notches and the inwardly protruding latch hooks latch onto the rear side of the chestpiece body 42, thereby retaining the retention ring 44 on the chestpiece body. In an alternative embodiment illustrated by FIG. 13, the chestpiece 12 may comprise a twist mechanism to secure the retention ring 44 onto the chestpiece body. Further, as one skilled in the art would understand, the retention ring 44 can be secured with alternative fastening mechanisms.

The diaphragm 40 suitably comprises a disc of silicone or other suitable material for auscultation. The diaphragm 40 comprises a peripheral edge margin. The diaphragm is sized and arranged to be positioned on the front side of the drum 46 such that the peripheral edge margin is supported by the rim 48. More particularly, the perimeter edge margin of the diaphragm 40 is configured to be captured (e.g., sandwiched) between the rim 48 of the drum 46 and the lip section 64 of the retention ring 44 when the retention ring is mounted on the chestpiece body 42 as described above. In particular, the lip section compresses or pinches the peripheral edge margin of the diaphragm 40 against the rim 48 to retain the diaphragm in a fixed position on the drum 46. When the diaphragm is retained between the drum 46 and the retention ring 42 in this fashion, the diaphragm 40 and the bell-shaped portion 50 of the drum define a space therebetween for receiving sound waves into the hollow passaging of the stethoscope 10.

As can be seen, the stethoscope 10 provides contiguous passaging between the diaphragm 40 and the ear tips 18 for auscultation. More particularly, the hole 52 and the radial lumen extending through the chestpiece stem 54 define a passaging section that provides fluid and acoustic communication from the space between the bell-shaped portion 50 of the drum 46 and the diaphragm 40 to the flexible extension tube 16. The flexible extension tube 16, in turn, defines a through passage or open-ended lumen that provides fluid and acoustic communication between the chestpiece 12 and the headset 14, in particular the stem lumen 26a of the headset. Further, the stem lumen 26a is configured to provide fluid and acoustic communication between the flexible extension tube 16 and each of the binaural lumens 26b, 26c, which in turn provide fluid and acoustic communication to the eartips 18 so that sound may be transmitted through the above-described stethoscope passaging from the diaphragm 40 to the ears of the medical practitioner using the stethoscope.

To manufacture the stethoscope 10, each of the parts is separately formed in a suitable manufacturing process. For example, each of the headset 14, the chestpiece body 42, the eartips 18, and the retention ring 44 is formed in an injection molding or additive manufacturing process. In one or more embodiments, the diaphragm 40 is stamped or cut from suitable sheet or film material. Likewise, the extension tube 16 may be cut from a longer length of flexible tubing. From the individual parts, the stethoscope 10 can be formed by attaching the eartips 18 to the eartip mounts 23, 25, sandwiching the perimeter edge margin of the diagram 40 between the retention ring 44 and the chestpiece body 42 and latching the components together as described above, and connecting the extension tube 16 between the chest piece 12 and the headset 14 (e.g., by inserting the stems 20, 54 into the open ends of the extension tube to attach the components together by friction fit).

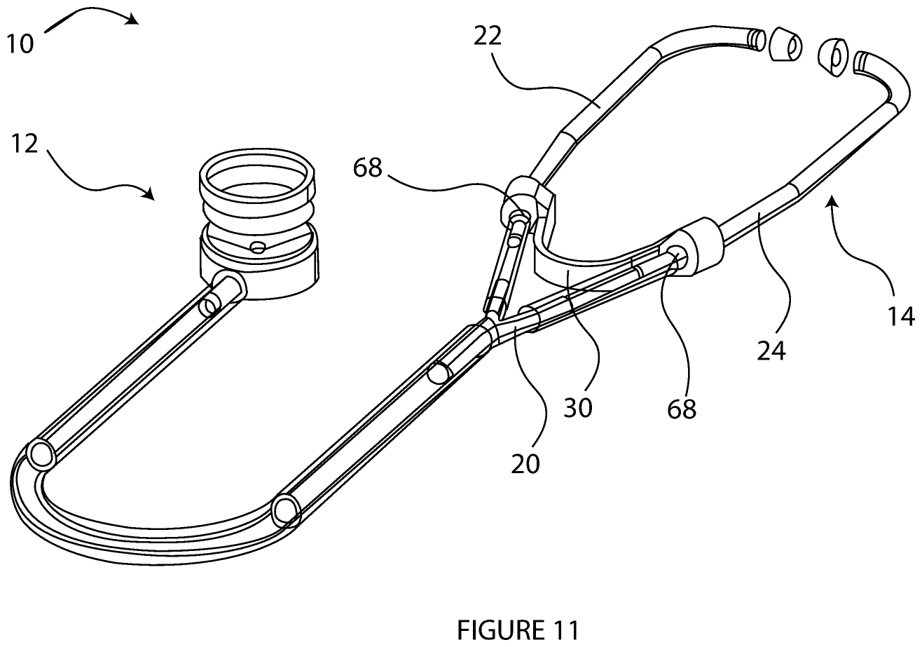

Referring to FIG. 11, in another embodiment of a stethoscope 10, the headset 14 is not a unitary piece of material, but instead comprises several connectable pieces. The headset 14 shown in FIG. 11 comprises separate binaural tubes 22, 24, a separate brace 30 connected to each of the binaural tubes 22, 24, and a wye-shaped stem 20. In the present embodiment, the brace 30 is generally U-shaped and comprises holes 68. The binaural tubes 22, 24 extend through the holes 68 of the brace 30 such that the brace is configured to support the first and second binaural tubes 22, 24 in a laterally spaced apart relation. The stem 20 has two laterally spaced upper end portions and a lower end portion. The stem upper end portions are connected to the lower end portions of the binaural tubes 22, 24 and the lower end portion of the stem is connected to the chestpiece 12 by extension tubes.

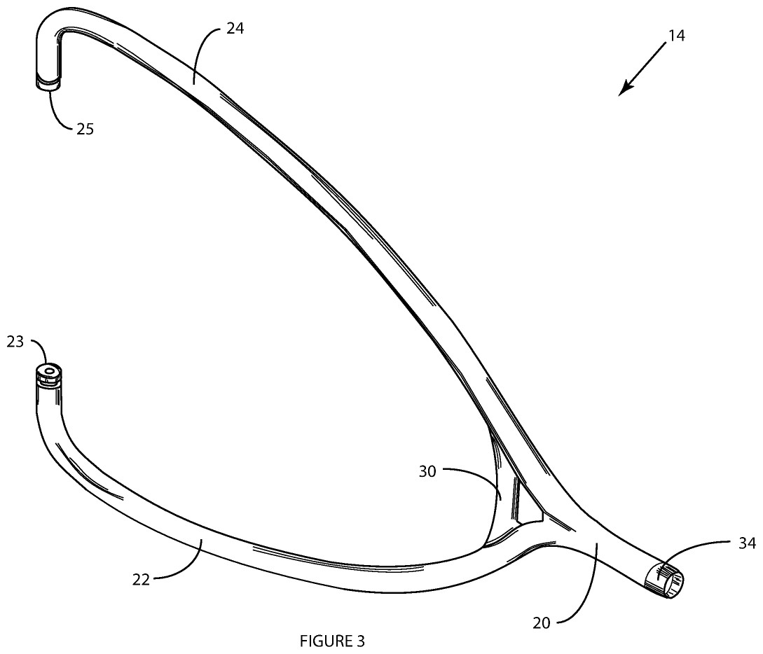

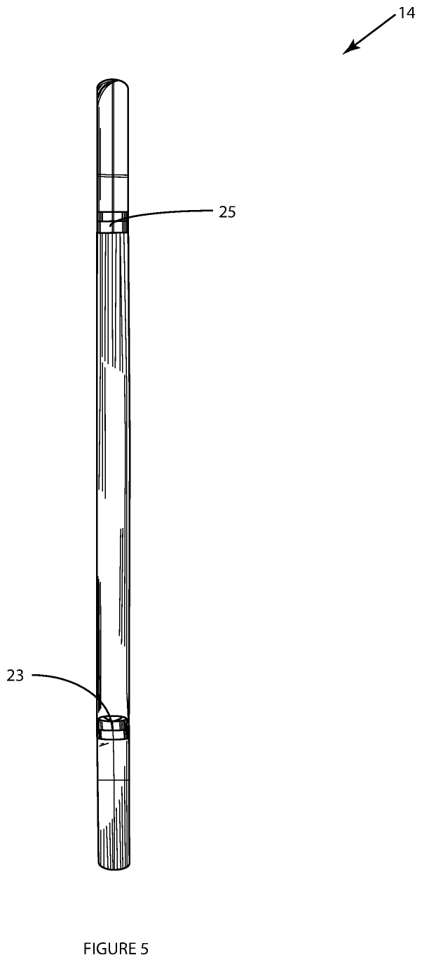

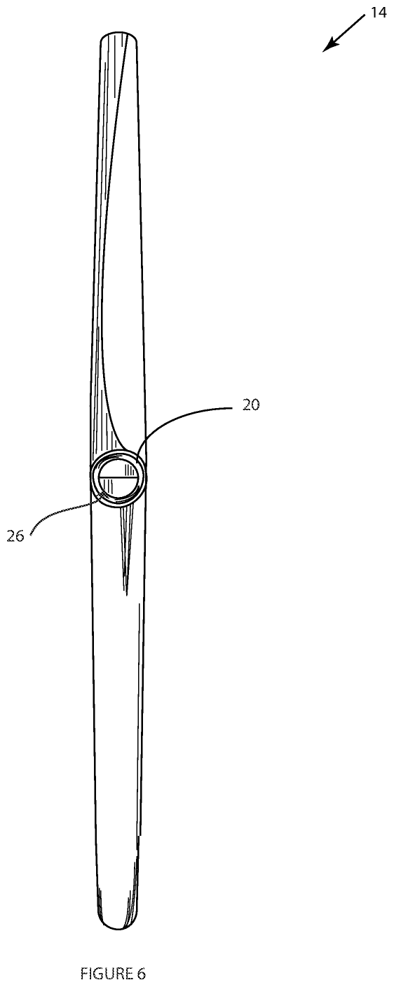

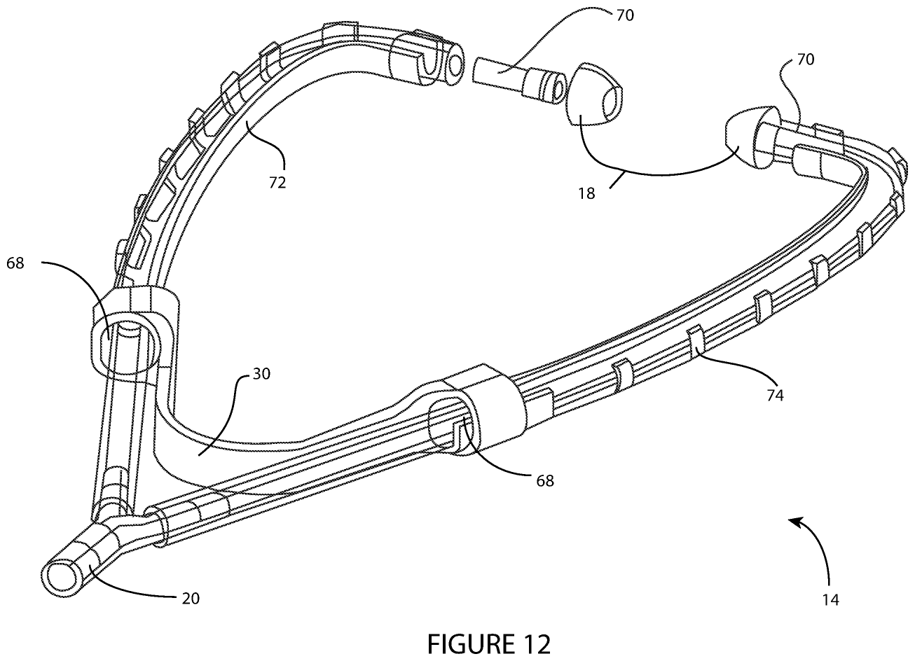

Yet another alternative embodiment of the headset 14 is shown in FIG. 12 in which the headset comprises eartip connectors 70, a pair of tubing supports 72, 74, the brace 30, and the stem 20. Each tubing support 72, 74 is configured to form a cradle around a respective binaural tube and to hold the respective binaural tube in a bent configuration suitable for use in the head set. It can be seen that the tubing supports 72, 74 enable pliable lengths of tubing that are not resiliently biased into a headset shape to be held in the desired shape. (by contrast, in FIGS. 1-10 the binaural tubes are formed of resilient material to hold their shape). The tubing supports 72, 74 may be connected to the brace 30. The upper portions of the stem 20 each connect to the lower portions of the extension tubes, which then extend through the holes 68 in the brace 30. The eartip connectors 70 connect the upper portions of the extension tubes to the eartips 18.

The foregoing description has been presented for the purposes of illustration and description. It is not intended to be exhaustive or to limit the disclosure to the precise form disclosed. Many modifications and variations are possible in view of this disclosure. Indeed, while certain features of this disclosure have been shown, described and/or claimed, it is not intended to be limited to the details above, since it will be understood that various omissions, modifications, substitutions and changes in the apparatuses, forms, method, steps and system illustrated and in its operation can be made by those skilled in the art without departing in any way from the spirit of the present disclosure.

Furthermore, the foregoing description, for purposes of explanation, used specific nomenclature to provide a thorough understanding of the disclosure. However, it will be apparent to one skilled in the art that the specific details are not required in order to practice the disclosure. Thus, the foregoing descriptions of specific embodiments of the present disclosure are presented for purposes of illustration and description. They are not intended to be exhaustive or to limit the disclosure to the precise forms disclosed, many modifications and variations are possible in view of the above teachings. The embodiments were chosen and described in order to best explain the principles of the disclosure and its practical applications, to thereby enable others skilled in the art to best utilize the disclosed system and method, and various embodiments with various modifications as are suited to the particular use contemplated.

What is claimed is:

1. A disposable stethoscope comprising:
a headset; and
a chestpiece assembly configured to be acoustically connected to the headset for conveying sound to the headset, the chestpiece assembly comprising a diaphragm, a chestpiece body, and a retention ring; the chestpiece body comprising a drum having a front side and a rear side spaced apart along a drum axis, the front side of the drum including a peripheral annular rim and the drum comprising a central hole opening through the front side of the drum; the diaphragm comprising a peripheral edge margin, the diaphragm being configured to be positioned on the front side of the drum such that the peripheral edge margin of the diaphragm is supported on the rim; the retention ring comprising a ring portion extending circumferentially about a ring axis and at least one latch arm extending along the ring axis to a respective free end portion, the retention ring being configured to be positioned on the chestpiece body such that the retention ring sandwiches the peripheral edge margin of the diaphragm between the rim and the ring portion and the at least one arm latches with the chestpiece body to attach the retention ring to the chestpiece body and thereby retain the diaphragm on the drum.

2. A disposable stethoscope as set forth in claim 1, wherein the disposable stethoscope further comprises a first eartip and second eartip, the first and second eartip being configured to be acoustically connected to the chestpiece via the headset.

3. A disposable stethoscope as set forth in claim 2, wherein the headset comprises a generally wye-shaped tubing.

4. A disposable stethoscope as set forth in claim 3, wherein the wye-shaped tubing of the headset further comprises a stem tube, a first binaural tube, and a second binaural tube, wherein the stem tube further having a lower end portion and an upper end portion, wherein the first binaural tube extends upward from the upper end portion of the stem tube in a first direction to a first binaural end portion, and wherein the second binaural tube extends upward from the upper end portion of the stem tube in a second direction to a second binaural end portion.

5. A disposable stethoscope as set forth in claim 4, wherein the first binaural end portion defines a first eartip mount, wherein the second binaural end portion defines a second eartip mount, and wherein the first and second eartip mounts mount the first and second eartips, respectively.

6. A disposable stethoscope as set forth in claim 5, wherein the headset further comprises a brace extending laterally from a first brace end portion connected to the first binaural tube at a location spaced apart between the upper end portion of the stem tube and the first binaural end portion to a second brace end portion connected to the second binaural tube at a location spaced apart between the upper end portion of the stem tube and the second binaural end portion such that the brace is configured to support the first and second binaural tubes in laterally spaced apart relation.

7. A disposable stethoscope as set forth in claim 6, wherein the headset defines passaging providing communication from the lower end portion of the stem to each of the first and second binaural end portions.

8. A disposable stethoscope as set forth in claim 7, wherein the passaging includes a stem lumen extending along the stem from a lower end portion opening through the lower end portion of the stem to an upper end portion opening through the upper end portion of the stem, a first binaural lumen extending along the first binaural tube from a lower end portion connected to the upper end portion of the stem lumen to an upper end portion opening through the eartip mount of the first binaural tube, and a second binaural lumen extending along the second binaural tube from a lower end portion connected to the upper end portion of the stem lumen to an upper end portion opening through the eartip mount of the second binaural tube.

9. A disposable stethoscope as set forth in claim 8, wherein each of the first and second binaural lumens has a cross-sectional dimension that tapers as the respective binaural lumen extends upward from adjacent the respective lower end portion toward the respective upper end portion.

10. A disposable stethoscope as set forth in claim 9, wherein the headset is formed from a single piece of monolithic material.

11. A disposable stethoscope as set forth in claim 9, wherein the stethoscope is formed from a plurality of connectable pieces.

12. A disposable stethoscope as set forth in claim 11, wherein the connectable pieces comprise separate first and second binaural tubes, separate brace connected to each of the first and second binaural tubes, and a separate wye-shaped stem connected to each of the first and second binaural tubes.

13. A disposable stethoscope as set forth in claim 12, wherein the brace is generally U-shaped and further comprises holes, wherein the first and second binaural tubes extend through the holes of the brace such that the brace is configured to support the first and second binaural tubes in a laterally spaced apart relation.

14. A disposable stethoscope as set forth in claim 13, wherein the stem has two laterally spaced upper end portions and a lower end portion, wherein the stem upper end portions are connected to the lower end portions of the first and second binaural tubes and the lower end portion of the stem is connected to the chestpiece by extension tubes.

15. A disposable stethoscope comprising:
a chestpiece;
first and second eartips; and
a headset configured to acoustically connect the chestpiece to the first and second eartips for transmitting sound from the chestpiece to the first and second eartips, the headset comprising generally wye-shaped tubing including a stem tube having a lower end portion and an upper end portion, a first binaural tube extending upward from the upper end portion of the stem tube in a first direction to a first binaural end portion, and a second binaural tube extending upward from the upper end portion of the stem tube in a second direction to a second binaural end portion, each of the first and second binaural end portions defining an eartip mount configured to mount a respective one of the first and second eartips on the headset, the headset further comprising a brace extending laterally from a first brace end portion connected to the first binaural tube at a location spaced apart between the upper end portion of the stem tube and the first binaural end portion to a second brace end portion connected to the second binaural tube at a location spaced apart between the upper end portion of the stem tube and the second binaural end portion such that the brace is configured to support the first and second binaural tubes in laterally spaced apart relation;
wherein the headset is formed from a single piece of monolithic material.

16. A disposable stethoscope comprising:
a chestpiece;
first and second eartips; and
a headset configured to acoustically connect the chestpiece to the first and second eartips for transmitting sound from the chestpiece to the first and second eartips, the headset comprising generally wye-shaped tubing including a stem tube having a lower end portion and an upper end portion, a first binaural tube extending upward from the upper end portion of the stem tube in a first direction to a first binaural end portion, and a second binaural tube extending upward from the upper end of the stem tube in a second direction to a second binaural end portion, each of the first and second binaural end portions defining an eartip mount configured to mount a respective one of the first and second ear tips on the headset;
wherein the headset defines passaging providing communication from the lower end portion of the stem to each of the first and second binaural end portions;
wherein the passaging includes a stem lumen extending along the stem from a lower end portion opening through the lower end portion of the stem to an upper end portion opening through the upper end portion of the stem, a first binaural lumen extending along the first binaural tube from a lower end portion connected to the upper end portion of the stem lumen to an upper end portion opening through the eartip mount of the first binaural tube, and a second binaural lumen extending along the second binaural tube from a lower end portion connected to the upper end portion of the stem lumen to an upper end portion opening through the eartip mount of the second binaural tube; and
wherein each of the first and second binaural lumens has a cross-sectional dimension that tapers as the respective binaural lumen extends upward from adjacent the respective lower end portion toward the respective upper end portion.
